(19) United States
(12) Patent Application Publication
(10) Pub. No.: US 2021/0299656 A1
(43) Pub. Date: Sep. 30, 2021

(54) Magnetic Nanoparticle Distribution in Microfluidic Chip

(71) Applicant: National Research Council of Canada, Ottawa (CA)

(72) Inventors: Lidija MALIC, Montreal (CA); Liviu CLIME, Longueuil (CA); Daniel BRASSARD, Longueuil (CA); Xuefeng ZHANG, Boucherville (CA); Teodor VERES, Montreal (CA)

(73) Assignee: National Research Council of Canada, Ottawa, ON (CA)

(21) Appl. No.: 17/265,292

(22) PCT Filed: Aug. 2, 2019
(86) PCT No.: PCT/IB2019/056616

§ 371 Date: Feb. 2, 2021

Related U.S. Application Data
(60) Provisional application No. 62/714,391, filed on Aug. 3, 2018.

(51) Int. Cl. B01L 3/00; H01F 1/33; B03C 1/025

(52) U.S. Cl. CPC B01L 3/502707; H01F 1/33; B03C 1/025; B82Y 25/00; B01L 3/50273; B01L 3/502761; B01L 3/502715

(57) ABSTRACT

The present invention relates into a device and method for controlling distribution of superparamagnetic nanoparticles (NPs) in a microfluidic chamber. By applying a strong magnetic field, localization of the NPs to inter-pillar spaces between soft magnetic coated micropillars is demonstrated, even with a modest fluid flow across the inter-pillar space. Flow splitting techniques are also provided to force particles to reliably interact with the NPs, specifically by using a Brevais lattice with a primative vector of 1°-15° with respect to flow direction. The pillars may have non-circular cross-sectional shape and be arranged to direct NP clouds more effectively. An array of the pillars has multiple axes for rotating NP cloud distributions in multiple orientations, allowing for a rotating magnetic field to move the NP cloud for mixing a fluid that is otherwise stationary.

### FIELD OF THE INVENTION

The present invention relates in general to a technique for controlling magnetic nanoparticle distribution in a microfluidic chamber, and in particular to a kit, method and system for constraining magnetic nanoparticles to within spaces between micropillars.

### BACKGROUND OF THE INVENTION

Microfluidic devices offer important opportunities for controlled movements of fluids. Tiny volumes of fluids are advantageous when small amounts of samples or reagents are available, where compact or portable assays are needed, where automation is essential for efficiency, and where fast reaction times are sought, especially in clinical diagnosis and biomedical research. A variety of capillarity, centrifugal, pneumatic, and electrostatic microfluidic devices have been provided to move fluids and perform various types of biochemical assays (which is to be understood broadly, and to include at least Lab on Chip (LOC), micro-total analysis systems (μTAS), organ on chip, and other assaying devices for processing fluid in volumes of less than 10 mL, such as from 10 nL-1 mL). The most basic challenge in microfluidics in general, is to manipulate fluids in a controlled, repeatable manner, to achieve a desired process.

In this context, magnetization of particles can be very useful for controlling interactions between particles and a fluid. For example, immunomagnetic separation is a widely recognized approach for sample preparation.¹⁻³ Separation of target species, such as cells, may be achieved using immunomagnetic particles (i.e. magnetic nanoparticles conjugated to capture probes, such as antibodies) that bind specifically to cell surface receptors. Conventional immunomagnetic separation is typically performed in tubes with several commercially available kits, including MACS® system (Miltenyi Biotec, USA), CellSearch System (Veridex, USA), and Dynabeads MPC separator series (Life technologies, USA). Furthermore, many applications call for chemical interaction of samples with functionalized superparamagnetic particles for sensitive detection of analytes, including food- and water-borne testing, blood testing, pharmacological testing, and clinical and biological testing.

In the past decade, microfluidic-based approaches that leverage magnetism have also emerged as viable, high throughput, low cost alternatives.^{4,5} “When brought into a microfluidic channel, nano- and micro-particles offer a relatively large specific surface for chemical binding.”⁴ Open or “empty” microchannels in microfluidic structures can be loaded with packed beds of functionalized particles or particle suspensions to profit from an even larger surface-to-volume ratio, an enhanced interaction of reactive surfaces with fluids passing by, and an improved recuperation of reaction products.⁴ Thus packed beds and bead suspensions are both known in the art. Packed beds may be porous enough to offer relatively low resistance to the flow while retaining the particles well enough to prevent (or reduce to a satisfactory level) their entrainment in the flow. However, the engineering required to achieve these is non-trivial.

Using magnetic nanoparticles MNPs offers some advantages over larger particles. Herein a MNP refers to nanoparticles: with a size in the range of 1-500 nm, preferably in the range of 5-250 nm or 20-200 nm; having magnetic moment per unit mass of 0.5 to 1 Am²/Kg, such as about 0.67 Am²/Kg and a saturation field of about 100-1000 kA/m, such as 500 kA/m, as can be produced with one or more superparamagnetic domains within each particle; and having a surface that is effective to avoid agglomeration (such as via electrostatic or steric repulsion). MNPs have higher surface areas, for higher interaction potential, no magnetic remanence and lower inertia and hydrodynamic drag, for fast response in a fluid.

It would be desirable in the art to improve control over spatial distributions of MNPs so that smaller amounts of nanoscale powders can be effective to interact with suspended or dissolved species within a microfluid. While the nanoparticles can be distributed randomly within a magnetic field, there is limited ability to control distribution and movement of the beads, because of the substantial limits on spatial and temporal variation of the magnetic fields within microfluidic chambers.

Given the complexity of magnetic field generators and their control apparatus; the need to vary, apply and remove the magnetic field during microfluidic processes; and the preference for avoiding contamination and cleaning issues by reusing microfluidic chips (which can otherwise be made very inexpensively), it is cost effective to avoid integration of controllable magnetic field generators within the chip. Most microfluidic methods rely either on (i) positioning a magnet in the vicinity of the microfluidic channel^{6,7} where the magnet provides both magnetic field and magnetic field gradient for the magnetic capture process, or (ii) using a magnet as a field generator and soft magnetic elements integrated into (or in the immediate vicinity of) the channel for the magnetic field gradient (high gradient magnetic separation—HGMS).

U.S. Pat. No. 7,601,265 to Rida et al. (Rida) teaches methods for manipulating magnetic micro-/nano-particles with magnets. The embodiments of FIGS. 1-17 concern a flow-through reactor in the form of a tube. Embodiments of FIGS. 18-20 address a simplistic “microchip like structure” that consists of a single microchannel. Rida notes a challenge with respect to maintaining a very localized high magnetic field gradient necessary for manipulating magnetic particles, on a microscopic scale in chips. Rida teaches ferromagnetic material sheets snuggly fitted into openings of the chip layer defining the microchannel. This allows a large electromagnet to be registered in position and drive a magnetic field across the microchannel. The ferromagnetic material sheets have a toothed structure in order to produce a pattern of regions of high and low magnetic field amplitudes that permit the magnetic particles to be arranged to form a periodic distribution of chains of magnetic particles. Rida teaches that by applying an AC current of a sufficiently high frequency to a winding of the electromagnet a “vortex rotational dynamic” is produced that is said to be advantageous.

Rida teaches that chains of superparamagnetic particles are densified and held together. A resulting risk is obstruction of flow through the tube or microchannel, which is obviously problematic for achieving high surface area required for high fluid interaction potential. The manipulation of magnetic particles is challenging for flow through arrangements of the magnetic particles, because too strong a flow tends to result in loss of particles, and too strong a magnetic field reduces fluid permeability, and does not necessarily provide a high surface area.

By temporally varying the magnetic field (high frequency AC), Rida teaches agitating the “chains” of particles to produce a “vortex rotational dynamic” that provides a more efficient homogeneous distribution of the magnetic particles over the cross-section of the flow channel, even with a lower density of magnetic particles, and permits a more efficient interaction between the magnetic particles and target particles carried by a liquid flowing through the cell. Rida still requires a fairly high number of particles, and is limited by the spatial and temporal control over magnetic fields within microfluidic channels.

It appears that Rida has no appreciation for the effect that use of MNPs (as opposed to larger particles in the nanometer or micrometer range), can have on the ability to produce chains thereof, given that they disclose (C1;L50) that any magnetic particles can be used. The Sinclair article referred to by Rida reveals that all particles are micron-sized with the one exception: “Miltenyi Bioteck manufactures the smallest beads on the market—a mere 50 nm in diameter.” C6,L12 of Rida shows a preference for magnetic particles with a diameter of 2-5 microns.

It will be appreciated that using larger diameter particles (micron scale) with high magnetic moments will enable effective control over the particles with weaker external fields ceteris paribus, but at the cost of efficiency of binding with targets. It is known that when trapping micron scale targets, such as cells and bacteria, micron-scale particles do not provide sufficient surface area to volume capture area, and mobility to provide sufficient interaction probability with a sample stream. Thus nanoparticles are preferred, but these are harder to control magnetically. The corrugated poles provide the limit of control of magnetic field gradients available, without some kind of magnetic bodies within microfluidic channels of microfluidic devices.

High gradient magnetic separation (HGMS) is a field of study most closely related to the present invention. Proposed HGMS approaches to improve magnetic gradients within microfluidic chip include embedding patterned soft magnetic materials in microscopic elements to create local distortions of an externally applied magnetic field and thus generate stronger magnetic gradients. These can, for example, be used for efficient separation of magnetic vs. non-magnetic targets in microfluidic devices, or for immunomagnetic separation. HGMS devices typically use ferromagnetic wires for their desirable dimensions, and their demonstrable ability to create strong magnetic gradients when subjected to external magnetic fields.⁸ For example, the device described by Inglis et al. used ferromagnetic stripes recessed into a silicon substrate to alter the flow of magnetically labeled cells by magnetizing the stripes by an externally applied field.^{9,10} Magnetically labeled cells were attracted to the strips and tended to follow the strip direction, while unlabeled cells did not interact with the strips and followed the direction of fluid flow. Magnetic species can be trapped in separated channels by the high gradient magnetic regions created by arrays of small wires.¹¹ Finally, repulsive modes in HGMS devices can also be used for diamagnetic targets.¹²

One of the problems with HGMS devices reported in the literature relate to the difficulties in releasing (cleaning) captured magnetic particles¹³, and in particular, the difficulty in preventing the magnetically labeled particles (cells) from permanent adhering to the magnetic elements. In the device designed by Inglis et al., a significant number (˜50%) of magnetically labeled cells either stuck permanently to the nickel strips or were not sufficiently attracted to the stripes to be separated.^{9,10}

To alleviate this problem, pillars on silicon substrates for HGMS have been formed of permalloy or other soft magnetic materials.¹⁴ A good trade-off has not been found between strong enough magnetism for effective retention of magnetically labelled particles, and timely release thereof thereafter.

For complex biological assays, the release of magnetically labelled particles is highly desirable for further downstream processing. However, the reported devices had difficulty releasing captured material due to the device design that is focused on creating magnetic field gradients as high as possible to maximize the capture forces, using solid magnetic wires²¹⁻²³ as they provide maximum perturbation effects and non-uniformities (gradients) in the applied field. However, in addition to the poor control of the capture regions due to an attractive capture force present everywhere on the surface of the wires/pillars,²⁴ the large amounts of magnetic material employed usually possess strong remnant magnetization. This creates significant capture forces that persist after removal of the external magnetic field which makes the release challenging.

While very high flow rates and associated drag forces may improve release of trapped particles, the removal in low flow regions and stagnation points on the pillars are particularly challenging, especially using only a single unidirectional liquid flush. Various strategies, such as coating the magnetic material with PDMS,¹⁸ have been employed to improve magnetic release, which further complicates the process of making these MNPs, and increases the cost of these devices.

Magnetizable nickel coated posts in microfluidic channels have advantages over solid wires. Deng et al. showed a simple process based on electrodeposition of Ni integrated with PDMS microfluidic devices.¹⁷ The same process was later employed by Yu et al., and Liu et al., to integrate Ni pillars for magnetic capture of cells.¹⁸⁻²⁰ Specifically, microtransfer molding of a PDMS chip was used¹⁷ to form the posts, and electrodeposited nickel coatings were applied to the posts (7 μm high and 15 μm in diameter, or an aspect ratio of 0.5:1). After the nickel coatings were deposited, an external permanent magnet was used to magnetize the posts. The magnetic field generated was ˜40 kA/m. Both transverse and axial magnetic fields were used. The device is proposed for trapping MNPs and separation of MNPs from a fluid.

While this could potentially decrease the fabrication costs, the process suffers from low-throughput. In addition to the difficulty of integrating soft magnetic materials in microfluidic channels, the devices reported in the literature suffered from small channel sizes and low density of magnetic microstructures.¹⁷⁻²⁰ This typically results in low flow rates, low MNP capture capacity and limits use in higher throughput applications where a large number of magnetic beads have to be processed.

A more recent disclosure by Applicant in 2015²⁵ teaches magnetizable nickel-coated pillars in microfluidic channels for producing magnetic field variations that form MNP capture and MNP depleted regions. Higher aspect ratio nickel coated pillars (3:1 prior to coating and ˜3.5:1 after coating) with thinner (2 μm) nickel coating, and a 100 kA/m field were shown to produce magnetic field gradients in a denser array of pillars, suitable for trapping nanoparticles. Local magnetic fields surrounding the pillars are examined and mapped out, and modeling shows depletion and capture areas. While this disclosure shows a possibility for using field variations from magnetizable arrayed pillars, FIG. 3(c) therein explains that a substantial collapse of both the depleted regions and capture regions is observed under moderate fluid flow conditions with MNPs.

Accordingly there remains a need for improved techniques for controlling MNP distribution in microfluidic chambers of microfluidic chips, and in particular to a method of distributing the MNPs in a spatially constrained region that extends between the magnetic integrated in the microfluidic chamber, especially one that maintains the distribution while a sample is flowed across the region. The need remains for a better trade-off between magnetic capture strength, and quick and reliable demagnetization for release. By flowing a sample through a MNP cloud of this distribution, improved interaction with the MNPs is possible with: lower incubation time, higher capture efficiency, using fewer MNPs, or under higher flow rates of the sample.

### SUMMARY OF THE INVENTION

Applicant has demonstrated that with a dense, high aspect ratio array of pillars coated with magnetizable material, arranged in rows within a microfluidic chamber, the rows aligned with an externally generated magnetic field of sufficient strength, distribution of MNPs to form a cloud region substantially limited to row spaces between the pillars, that a density of the MNPs across this space is sufficient that there are no visible gaps in the cloud between the row's pillars, and that no visible gaps appear, even when subjected to moderate flow thereacross.

In some embodiments of the invention, a process for forming such a chamber in a microfluidic chip, with improved cost effectiveness, is provided by producing the pillars on an insert, and bonding the insert in a microfluidic chip.

In some embodiments of the invention, the coated pillars are used both as magnetic capture features, for creating field gradients to control MNP cloud distribution, as well as fluidic obstacles, to distort fluid streamlines, and force the biological target species to interact with the functionalized nanoparticles in the nanoparticle cloud. By aligning the rows of pillars to a small angle with respect to stream lines, a controlled interaction with particles of a given range of sizes and densities can be preferentially induced into crossing through the cloud regions. Furthermore, as particles passing through the cloud regions are moving substantially parallel with the cloud regions, a dwell time within the cloud region is increased compared with substantially orthogonal traversal. Even within the cloud region, differences in MNP concentrations are expected. Where the flow is highest (furthest from the pillars, floor and ceiling, or near the centre) is also the most depleted of MNPs. The trajectories of particles in the stream in this layout discourages flow through the depleted regions, and favours flow through higher MNP concentration paths. This may reduce time and complexity of the assay by avoiding the need for diffusion-based incubation and mixing.

By controlling a thickness of the magnetic material coating on the pillars, remanence of the magnetic field can be low, allowing for a satisfactory magnetic field surrounding the pillars, with suitable depletion and capture areas, but also providing for a fast and reasonably complete release of the particles in a moderate flow once the magnetic field is removed.

By providing an array of the rows, each row having a respective alignment with respect to the others with a respective stagger, the magnetic field being movable into position to alignment with a plurality of different rows in different directions. The different rows in different directions can consist of all of the same pillars, or different overlapping subsets of pillars can be used for different directions. By arranging for two or more differently directed rows, and by moving external magnets between different aligned positions, or moving different magnets or sets of magnets towards and away from the chamber, a rapid movement of the MNPs can be performed to permit mixing or interaction without any other fluid motion. Thus magnetic stirring can be performed with enhanced interaction probability, in what is otherwise a stagnation chamber.

A copy of the claims as filed are incorporated herein by reference.

Accordingly controlling superparamagnetic nanoparticle distribution in a microfluidic chamber of a microfluidic chip is provided, where: at least one row of at least 3 magnetically coated pillars are provided in a wall of the chamber, the pillars having a minimum separation with neighbors of 0.2-500 μm, an aspect ratio greater than 2:1, and a mean diameter of 1-1000 μm, where a polyline connects centres of the pillars; and a fluid is contained in the chamber surrounding the pillars, the fluid suspending superparamagnetic nanoparticles (NPs) that are self-repellant to reduce agglomeration. The control is provided by: applying a magnetic field to the chamber using magnets that are outside of the microfluidic chip, the magnetic field having a local field line that is at least 75% aligned with each segment of the polyline, wherein the NPs, pillars, and thickness of the magnetic coating of the pillars, are selected so that the NPs are substantially distributed between the pillars in that at least one of the following obtains: a NP density at every point between two adjacent pillars of a single row is at least 50% higher than the NP density midway between two adjacent rows; a NP density at every point between two adjacent pillars of a single row is at least 50% higher than the NP density a distance normal to the polyline equal to a mean separation of the pillars; a mean NP density in inter-pillar spaces between adjacent pillars is at least 10 times higher than a mean NP density within the chamber; a magnified view from a direction in which end faces of the pillars are in view, there are no visible gaps in the NP density between adjacent pillars of a single row, and visible gaps across at least 80% of the chamber away from the rows.

Preferably at least ⅓ of the NPs have a surface or subsurface coating for electrostatically, sterically, or chemically repelling like particles, and the NPs are surface functionalized to selectively bond to a target analyte. Preferably the NPs are distributed substantially only between the pillars in that at least 80% of the NPs are retained within one or more strips centred on the polylines, with a strip thickness of twice a mean diameter of the pillars.

The pillars may be coated with one of: a soft magnetic shell of thickness of 0.1-20 μm, composed of a nickel-based alloy; and a soft magnetic shell of thickness of 0.1-20 μm, composed of a nickel-based alloy coated with a gold passivation layer.

Controlling may further comprise flowing a sample fluid through the chamber across the NP distribution for NP analyte capture while the magnetic field is applied. If so, the wall may include at least 3 rows that form a two-dimensional Bravais lattice of the pillars, with one of the primitive vectors of the lattice being oriented at an angle between 1° and 15° with respect to the liquid flow through the chamber. If so, the magnetic field may be oriented: in a direction that minimizes an inter-pillar space between adjacent pillars of row; in a direction of one of two primitive vectors of a two-dimensional Bravais lattice of defined by the at least one row; or in a flow direction through the chamber, which is oriented at an angle between 1° and 15° with respect to one of two primitive vectors of a two-dimensional Bravais lattice of defined by the at least one row.

Controlling may further comprise removing the magnetic field after the sample flowed through the chamber, and flushing the NPs to a detection chamber. Flushing may be accomplished only with fluid dynamics, and without magnetic guidance, or a density or spatial distribution of the NPs is increased within the detection chamber by mechanical, flow, magnetic or ultrasonic filtration.

The sample fluid, after flowing through the chamber, may travel through a second chamber bearing a respective wall with pillars and a fluid suspending at least one second NP distribution with NPs functionalized to selectively bond to at least one second analyte, where a single magnetic field applies fields across the chamber and the second chamber concurrently. The chamber and second chamber may be stacked horizontally, for example on separately bonded and aligned microfluidic chips.

The pillars may have a mean separation of 1-100 μm, an aspect ratio greater than 3:1, and a mean diameter of 10-300 μm, the NPs may be electrostatically charged to prevent agglomeration; the magnetic field may have a local field line that is at least 90% aligned with the segments of the polyline, and has a magnetic field strength of at least 110 kA/m across this local field line; and during the application of the magnetic field, the NPs may be distributed substantially only between the pillars in that at least 80% of the NPs are retained within one or more strips centred on the polylines, with a strip thickness of twice a mean diameter of the pillars.

The pillars may have a mean separation of 20-80 μm, an aspect ratio greater than 5:1, and a mean diameter of 20-150 μm; the NPs may be electrostatically charged to prevent agglomeration; the magnetic field may have a local field line that is at least 90% aligned with the segments of the polyline, and have a magnetic field strength of at least 110 kA/m across this local field line; and during the application, the NPs may be distributed substantially only between the pillars in that at least 85% of the NPs are retained within one or more strips centred on the polylines, with a strip thickness of twice a mean diameter of the pillars.

The at least one row of at least 3 magnetically coated pillars further comprises an array having at least 2 axes, along each of which axes the pillars are arranged at least one row of at least 3 pillars with a minimum separation with neighbors of 0.2-500 μm, further comprising applying the magnetic field alternately along the axes to redistribute the NPs.

Further, a microfluidic device is provided, the device comprising: a microfluidic chip with at least one wall of a microfluidic chamber, the wall supporting at least one row of at least 3 micropillars, where the micropillars of the row: are arrayed to form a polyline; have mean diameters of 1-1000 μm; have mean separations of 0.2-500 μm; have aspect ratios greater than 2:1; and are composed of a low susceptibility material coated with a soft magnetic material; a generator adapted to apply a magnetic field of at least 110 kAmp/m across the at least one row; and a support comprising a holder for the microfluidic chip in at least one prescribed position and orientation, and a registration feature for registering the generator in a position in which a field line of the magnetic field is at least 75% aligned with the polyline.

The microfluidic device may further comprise a sample introduction chamber, an analyte detection chamber, and a sample flush reservoir, the sample introduction chamber coupled to an ingress of the microfluidic chamber by an inlet channel, the microfluidic chamber coupled to the reservoir by an outlet channel, and the microfluidic chamber coupled to the detection chamber by a NP channel.

Each of the at least one wall of the microfluidic chamber, may be provided as an insert into an opening within a patterned microfluidic chip.

The soft magnetic coating comprises a soft magnetic shell of thickness of 0.1-20 μm, composed of a nickel-based alloy to ensure a low remanence.

The microfluidic device may comprise a plurality of the microfluidic chambers on one or more microfluidic chips, and the support comprises a holder for the one or more microfluidic chips in prescribed positions and orientations, and the registration feature registers the generator in a position in which one or more field lines of the magnetic field generated are at least 75% aligned with each of the respective polylines of the respective walls of the microfluidic chambers.

The at least one row of at least 3 magnetically coated pillars may comprise an array having at least 2 axes, along each of which axes at least one row of at least 3 pillars are arranged with a minimum separation with neighbors of 0.2-500 μm, the holder comprises a plurality of registration features for registering the generator in respective positions in which field lines of the magnetic fields are at least 75% aligned with the axes.

Further, a kit is provided, the kit comprising: the microfluidic device, and a fluid suspending superparamagnetic nanoparticles (NPs), the fluid being injectable into the microfluidic channel, wherein: the NPs are self-repellant to reduce agglomeration, and applying the magnetic field to the chamber with the magnet in registered position, with fluid in the microfluidic channel, substantially distributes the NPs between the pillars in that pillars in that at least one of the following obtains: a NP density at every point between two adjacent pillars of a single row is at least 50% higher than the NP density midway between two adjacent rows; a NP density at every point between two adjacent pillars of a single row is at least 50% higher than the NP density a distance normal to the polyline equal to a mean separation of the pillars; a mean NP density in inter-pillar spaces between adjacent pillars is at least 10 times higher than a mean NP density within the chamber; a magnified view from a direction in which end faces of the pillars are in view, there are no visible gaps in the NP density between adjacent pillars of a single row, and visible gaps across at least 80% of the chamber away from the rows.

A kit is provided comprising the microfluidic device, and a fluid suspending superparamagnetic nanoparticles (NPs), the fluid being injectable into the microfluidic channel, wherein the NPs: have a surface or subsurface coating that makes at least ⅓ of the particles electrostatically or chemically repel like particles; and are surface functionalized to selectively bond to an analyte.

In either kit, the microfluidic device may have a plurality of microfluidic chambers, and a plurality of fluids are provided each suspending respective NP that are surface functionalized for selectively bonding to respective analytes. The magnetic field may be oriented: in a direction that minimizes an inter-pillar space between adjacent pillars of row; in a direction of one of two primitive vectors of a two-dimensional Bravais lattice of defined by the at least one row; or in a flow direction through the chamber, which is oriented at an angle between 1° and 15° with respect to one of two primitive vectors of a two-dimensional Bravais lattice of defined by the at least one row.

Furthermore, a microfluidic chip insert is provided for insertion in a microfluidic chip to form a chamber, the insert comprising at least one wall for the chamber, the wall defining at least one row of at least 3 pillars, where the pillars: are arrayed to form a polyline; have mean diameters (d) of 1-1000 μm; have mean separations of 0.2-500 μm; have aspect ratios greater than 2:1; and comprise a soft magnetic coating; and the polyline meets an edge of each pillar where the extent of the pillar is d or greater.

The polyline may meet the pillars at points where the extent of the pillar is strictly greater than d. The insert may comprise two ledges perpendicular to the wall defining sidewalls of the chamber, and a flow direction defined between the two sidewalls, wherein the wall includes at least 3 rows of the pillars that form a two-dimensional Bravais lattice, with one of the primitive vectors of the lattice oriented at an angle between 1° and 15° with respect to the flow direction.

Further features of the invention will be described or will become apparent in the course of the following detailed description.

### BRIEF DESCRIPTION OF THE DRAWINGS

In order that the invention may be more clearly understood, embodiments thereof will now be described in detail by way of example, with reference to the accompanying drawings, in which:

FIG. 1A is a schematic illustration of a cross-section of a magnetic micropillar for use in a microfluidic chamber in accordance with an embodiment of the present invention;

FIG. 1B is a schematic illustration of a row of 3 micropillars according to FIG. 1A for use in a microfluidic chamber in accordance with an embodiment of the invention;

FIGS. 2A-F are schematic illustrations of respective alternate cross-sections of micropillars for use in a range of embodiments of the present invention;

FIGS. 3A,B,C are schematic illustrations of chambers with magnetic micropillars in accordance with three illustrative embodiments of the present invention;

FIGS. 4A-H are schematic illustrations of respective steps in a process for forming a chip, with a metallized insert defining micropillars within a chamber in accordance with an embodiment of the present invention, and for using a magnetic field generator to control distribution of MNPs loaded in the chip, and further performing capture therewith;

FIGS. 5A,B are schematic illustrations of dual use of micropillars as flow control elements for ensuring particles in a sample stream pass through the cloud regions, and for forming the cloud regions with the suitable magnetisable coating and arrangement with respect to the applied magnetic field;

FIG. 6 shows an embodiment of the present invention with the dual use micropillars embedded in a microfluidic device;

FIGS. 7A-E are five magnified images showing MNP particle distributions produced to demonstrate the present invention;

FIG. 8 is a panel of 6 images showing time lapse capture with a provisioned microfluidic chamber; and

FIG. 9 is a SEM micrograph showing dozens of MNPs adhered to a cell.

### DESCRIPTION OF PREFERRED EMBODIMENTS

Herein a technique is described for distributing MNPs to form a cloud region between magnetic micropillars of a microfluidic chamber in a microfluidic chip. Applicant has demonstrated that the cloud distribution can be maintained while a sample fluid passes through the cloud. Advantageously, the magnetic micropillars can be coated with a thickness of magnetic material that permits low remanence magnetic actuation.

FIG. 1A is a schematic illustration of a cross-section of micropillar 10 in accordance with an embodiment of the present invention. The micropillar 10 is made of a core 12 composed of a non-magnetic material, surrounded by a magnetic coating 14 made of a soft magnetic material, such as Ni, Ni iron alloys (e.g. permalloy), Si iron alloys, soft magnetic ceramics, or a combination thereof, preferably having a relative permeability greater than 50, more preferably greater than 100, most preferably 250-1000. The coating 14 has an average thickness t_{c}. While the cross-section shown has a uniform thickness (except for the pointed ends), the degree to which the coating is uniform depends on the specific forming process of the core and the coating, and a uniform coating thickness is not required, as long as the distribution of magnetic material is replicated with sufficient consistency to produce predictable magnetic field gradients within its vicinity. Thus the shape of the core 12 may not be of uniform thickness, to the extent that of the shape of the micropillar 10 is the same as the shape of the core 12. As electroless plating is an efficient technique for depositing nickel-based magnetic coatings on non-conductive materials and electroless plating produces nearly uniform thickness coatings, the drawings assume a uniform coating.

The shape of micropillar 10 and core 12 is an equilateral triangle, and an average circle C_{avg} of the micropillar 10 is shown having a diameter d_{avg}. For any shape, an average circumference can be calculated, for example by computing the radial coordinates of the perimeter about a centre of the micropillar 10, which will vary between two positive values minimum radius rₘ and maximum radius rₘ, and can be estimated by analysis of magnified images or by optical, mechanical, hydrostatic or hydrodynamic inspection.

The shape of the cross-section of the micropillar 10 can have substantially any form. The pillars do not absolutely have to have a constant cross-section shape, area or dimension as a function of height, and can generally taper slightly for easier demolding if that is the forming route for the micropillar cores 12. That said, a mass of the coating 14 at all elevations from a base of the micropillar 10 is uniform enough to provide continuity of the magnetic field. This mass is preferably distributed substantially completely around the core 12 (at least) at most elevations, to ensure good attachment of the coating 14. If electroless plating is used to deposit the coating 14, the mass deposited at every elevation is proportional to the perimeter at that elevation, and so a perimeter of the pillars may be reduced by less than 10% from base to highest elevation, even if tapered. If a higher taper is required, it can be achieved with a gradual transition of a core base having a more circular form and a top having shape with a higher perimeter to area ratio.

FIG. 1B is a schematic illustration of a row of 3 micropillars 10 of FIG. 1A, respectively labelled 10a,b,c in a wall 15 of a microfluidic chamber. The row is associated with a piecewise linear path (polyline 16) from centres (c) of adjacent micropillars 10. The microillars 10 have an identified separation between adjacent pillars (s₁₋₂ and s₂₋₃) which are different in the illustrated example, even though uniformly separated micropillars with a uniform pitch may be preferable. Herein micropillar spacing, defined by an average spacing sᵢ₋ⱼ between the ith and jth micropillar of the row, is in the range of 0.2-500 μm. Preferably the variance in the spacings is less than 10%.

As shown in FIG. 1B, radial dependency on the azimuth of the micropillars 10 can lead to a spacing s between adjacent micropillars that is shorter than the separation of their centres by different amounts, depending on an angle of the micropillars, and direction of the separation. Where separation is non-uniform, small separations s may be present where flow rates are highest, or, if flow rates are the same between all pairs of pillars, with pairs of pillars at which the polyline 16's segment is least aligned with the magnetic field. Preferably each polyline segment is 75% aligned with the magnetic field, such that an angle θ between the polyline segment and magnetic field is less than 22.5°, more preferably less than 80% aligned (θ<18°), more preferably less than 90% aligned (θ<9°). As such, adjacent polyline segments have angles of greater than 135°, more preferably, more than 144°, or more than 162°.

A space between the pillars is bounded by a cross-hatched area on a wall 15 and the pillars 10.

The micropillars 10 have uniform height h greater than 2 μm, and less than 2 mm, and an aspect ratio (AR) given by h:d_{avg} of 2:1 or greater, more preferably 3:1, 5:1 or even 10:1. The aspect ratio is important for providing high throughput with low hydrodynamic resistance, and increasing a volume of the space between the pillars as a fraction of the volume of the chamber. The pillars preferably extend between two opposite walls of the chamber. The high aspect ratio improves a uniformity of the magnetic field gradients.

Note that microfluidic rheology also plays a part in the preferred layouts of these micropillars 10: as flow through these micropillars 10 will be laminar, a velocity gradient will naturally form between the micropillars 10 with slowest flow nearest the micropillars 10, and fastest midway therebetween. By selecting an arrangement and profile of the microfluidic pillars 10, this gradient can be minimized, to improve capture efficiency. Thus while a flat bottom surface 15 of a wall of a microfluidic chamber is shown in FIG. 1B, other arrangements are equally practicable.

It is noted that a shape and distribution of the micropillars 10 can be chosen to improve control over the nanoparticle distribution between neighboring pillars. In general, the shape and the orientation of the pillars is designed to create “anchor” points for the nanoparticle regions: i.e. spots where high magnetic capture forces (high gradient fields) are coincident with the stagnation points in the microfluidic flow. This allows the nanoparticle regions to extend from one anchor point to another anchor point with minimum depletion caused by the flow between the pillars.

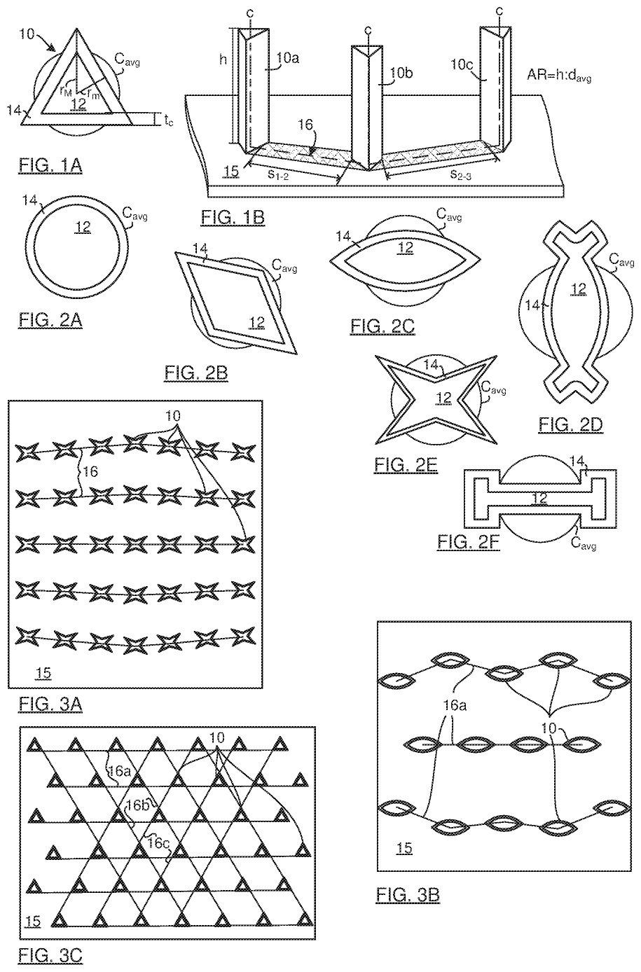

FIGS. 2A-F schematically illustrate cross-sections of a variety of micropillars 10 equally applicable in embodiments of the present invention, each schematically showing the average circle of the micropillar 10. FIG. 2A schematically illustrates an embodiment with a cylindrical pillar which has advantages for limiting hydrodynamic resistance, easy formation, and low sensitivity of alignment of the pillars with respect to each other (perfect radial symmetry). FIGS. 2B,C,D,E,F show elongated micropillars 10 that may preferably be arranged with long axes aligned with the magnetic field lines. The elongated structure is believed to allow for closer spacing between the anchoring points, with less volume to impeded flow; and also to increase coating magnetic material loading near the ends.

FIG. 2B and FIG. 2C illustrate that two lines can be replaced by a curve in any of the embodiments to achieve a slightly higher perimeter shape, and to generally reduce hydrodynamic resistance of the micropillar 10.

FIGS. 1A and 2A,B,C illustrate that a convex shape can be used, and FIGS. 2D,E,F, show that a concave shape can be used, the convex shapes generally having higher perimeter to volume. The cross-sectional shape of the pillar may have a greater surface area at two or more distal ends that face adjacent pillars in the array, such that more of the coating 14 material is concentrated at these ends than between these ends, as this may improve gradient magnetic field focus and resultant anchoring. FIGS. 2D,F and to some degree, FIG. 2E show a generally dog-bone shape with enlarged ends separated by a narrower midsection of greater length than the enlarged end. The elongated ends are believed to increase depletion and capture areas in preferred alignments of the micropillars (where the enlarged ends are proximal).

While the elongated structures of FIGS. 2B,C,D,F show essentially a two ended structure, each of the embodiments shown can be replicated with 3, 4, 5 or 6 ended structures, to match regular, or semi-regular array axes of the distribution of the micropillars in two or more rows. The micropillar cross-section of FIG. 2E has four acute vertices defining a rectangle: a major axis of the rectangle providing a higher separation of the opposite ends of the micropillar, with two separated pairs of corners. As such it is a 4 directed version of the 2 directed micropillar of FIG. 2B. The diagonals of the rectangle have symmetric tapered edges at opposite ends and each can define respective axes along which the micropillars may be arrayed. Alternatively (as shown below in FIG. 3A) both vertices of adjacent micropillars may be used in parallel to produce two parallel distributions between the two pairs of vertices of the two adjacent micropillars, assuming dense enough arrays can be produced with the correct gradient fields. An advantage to 3D arrangements on multiple axes is that changing alignment of externally applied magnetic fields, can allow for a redistribution of the cloud distributions, effectively switching of nanoparticle cloud distributions from one axis the other.

Approximations to any of these micropillar structures may be used, particularly those that are more easily patterned, more reliably patterned, or that exhibit least consequences to MNP distribution under applied field of imperfect forming.

FIG. 3A is an array of 5 rows of micropillars 10 according to FIG. 2E. Only four of the 35 micropillars 10 are identified and only two polylines of the 5 are identified, for clarity of illustration. A slight curvature of the polylines, and the row spacings of the micropillars 10 shown allows for a densification of the micropillars 10 near a centre of the chamber featuring the wall 15, assuming a flow from top to bottom, generally perpendicular to the polyline, while still providing for high alignment (over 90%) of the polylines to a magnetic field that is presumed to be uniform and oriented from left to right.

FIG. 3B is an array of 3 rows of micropillars 10 according to FIG. 2C. The polylines 16 include one linear polyline (100% aligned) and two non-linear polylines (more than 80% aligned; angle between polyline segments greater than 152°). The three polylines are parallel, having a same mean orientation, as is preferable, although a 10% variation (up to 9°) may be acceptable in some applications. This distribution is intended to illustrate that microfluidic rheological considerations may dictate an arrangement of the micropillars that is suboptimal for magnetic alignment. By interleaving micropillars on adjacent rows, a more homogeneous flow may be provided.

FIG. 3C is a regular array of 6 rows (on a first axis) of micropillars 10 according to FIG. 1A. As the micropillars 10 are triangular, it is convenient for a three-axis array, which happens to provide equally well defined polylines 16a,b,c on 3 different axes. Thus with a chamber defined with these features magnetized with north and south poles on the plane of the wall 15, rotating the poles with respect to the chamber results in aligning clouds of MNPs 6 times per revolution, which can facilitate interaction between a stationary fluid within the chamber and the MNPs. This chip design can also be used to vary magnetic fields during flow through processing if desired.

It will be noted that while each of the arrays of micropillars shown consist only of one kind (shape, size, orientation), and further that the spacing and arrangements have been exemplified by only a few arrangements, as long as the micropillars are of a satisfactory size, shape, and separation, and have sufficient soft magnetic material, they will collectively define capture and depletion regions that cooperate with adjacent capture and depletion regions of neighbouring micropillars, to permit (with the application of a sufficiently strong magnetic field) the retention of MNPs in a cloud configuration, to resist a modest fluid throughput.

FIGS. 3A,B,C schematically illustrate arrangements of micropillars 10 useful in forming confining MNP clouds to intra-row space between the micropillars. These arrangements are in at least part of a chamber of a microfluidic device, and in some embodiments, fill a complete cross-section of the chamber. The arrangements may be understood to be plan views of such a chamber. These figures may also be understood to be inserts for a microfluidic device, which may completely or partially define the wall 15 of the chamber. Furthermore inserts may comprise a plurality of such chambers and interconnecting channels. Trivially, an insert may be provided in a single chip, or a plurality of inserts may be provided in a single chip, for example in parallel or in series connection, or unconnected. Furthermore one or more inserts may be provided in two or more chips, and the chips may be overlaid with alignment of the chips. If two or more inserts are used in a same chip or chip set, both chips may have arrays with parallel axis, and are both magnetized by a same magnetic field, or alternatively each magnetization orientation may select at most one of the axes of one of the chips.

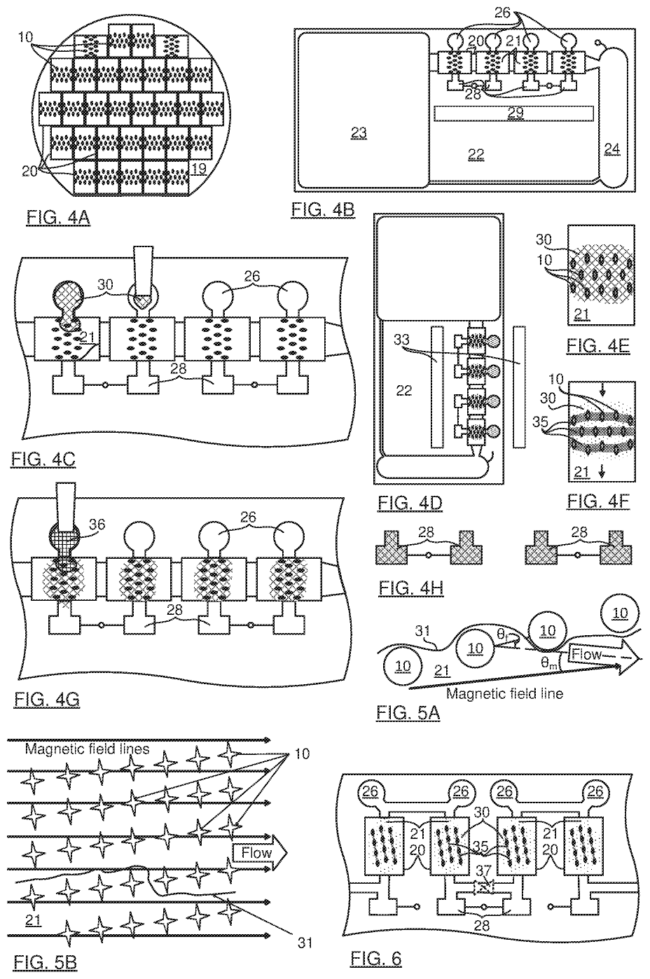

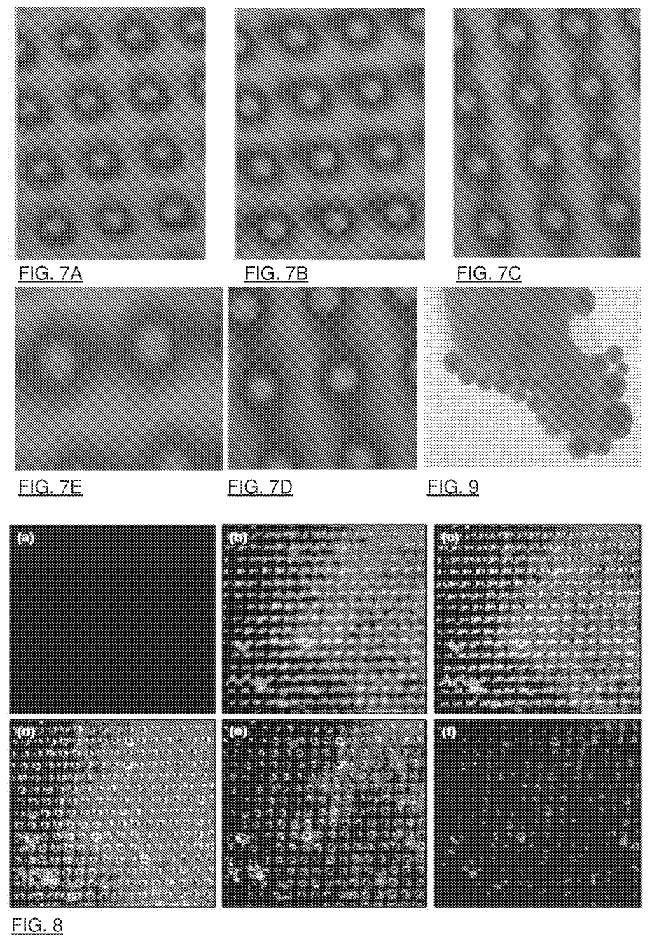

FIGS. 4A-H are a series of images at respective steps for manufacturing a chip, and using the chip for magnetic separation and analyte detection. At step 4A a substrate 19 is provided patterned with numerous instances of an insert 20 (only 4 labelled). While the illustrated embodiment shows 31 separately defined inserts 20, it will be appreciated that a uniform pattern of distributed micropillars 10 may be preferable, in some applications: if the pattern is symmetric under 90° rotation, dice lines may produce inserts running in either direction. The patterning involves at least defining one magnetic chamber 21 (only 4 labelled) having micropillars with a non-magnetic core that is coated with a magnetisable material. One of the patterned substrate 19, and a covering layer is advantageously composed of a thermoplastic elastomer (TPE) to permit low pressure, hermetic sealing with a variety of other surfaces, as explained in (PATENT 11804) whereby the diced substrate (insert 20) can readily bond to other parts of a microfluidic device. A hard polymer substrate such as COC, PC, would be used for the chamber and the pillars, if a film of TPE is used to seal the microfluidic device. This allows reliable fabrication of high aspect ratio pillars. The core and substrate may alternatively be composed of ceramic, glass, or low susceptibility metal. Advantageously, some parts of the substrate 19 may be masked to permit hermetic sealing with a chip and other layers of a microfluidic device, from both top surface and bottom surface.

An electroless plating process may be applied to metallize the substrate 19 with the soft magnetic metal coatings of previously described composition. Other coating techniques that form consistent distributions of the soft magnetic material can alternatively be used, including bath/immersion or solvent based deposition techniques similar to electroless plating with controlled surface adhesion, and mechanical insertion of coated non-magnetic rods or threads through the substrate 19 by a template, die, or registered machine. The former technique may offer better anchoring of the micropillars, while the latter techniques may avoid metallization of floor of the insert 20, reserving the metal for where it is needed for gradient field generation. The floor acts like an in-plane magnetized thin film, with minimal effect on the magnetic field within chamber. Magnetically isolated pillars can be produced by electrodeposition through porous membranes followed by gently removing (dissolving) the membranes afterwards. Side walls of the insert for the chamber may be defined in the insert, if alignment and integration with fluid paths of the chamber of the chip can be arranged. An advantage of coating side walls adjacent to the rows is the formation of cloud regions between pillars and the walls, and thus extending all the way across the chamber. In this way a large number (31 shown in the present example, but any other number is possible) of inserts 20 can be formed. The metallized substrate is diced to produce the inserts.

FIG. 4B schematically illustrates a microfluidic chip 22 having a sample prep(aration) area 23, the design of which is expected to depend on the application for which the chip 22 is designed. Between the sample prep area 23 and a waste reservoir 24, there is a 4 stage magnetic capture and release section of the chip 22. Four individual inserts 20 (only 2 labelled) are aligned and placed within respective openings in the chip 22. The inserts 20 are rectangular and symmetric, with sample inlets and outlets in the middle of narrow sides, and MNP inlets and outlets in the middle of long sides, respectively. The openings into which the inserts 20 are received are flanked by respective inlets 26 for loading MNPs opposite MNP detection chambers 28, and, in the adjacent, opposing, cardinal directions, by sections of a series path from the sample prep area 23 to the waste 24. Recesses in the chip 22 for receiving the inserts 20 form seals around the insert, but leave open access to the top, patterned surface thereof, and the micropillars 10 thereon. The capture chambers can also be connected in parallel.

A slot 29 is provided in the chip 22 for aligning a magnet with the inserts, such that magnetic field lines are substantially aligned with the rows of micropillars in each of the 4 magnetic chambers.

While irrelevant to the drawing, it will be appreciated that the chip 22 conventionally has a top cover bonded thereto, that would typically be transparent. If so the transparent cover has holes aligned with MNP loading inlets 26, or suitable puncture films, for loading via a syringe or dropper in one of the various ways known in the art. Likewise ports or air holes in fluid communication with the detection chambers 28 are open to ambience, or may be subjected to a negative pressure, in order to imbibe the MNPs in a fluid (typically liquid) carrier 30.

It will be appreciated that other routes for producing a magnetic chamber 21 in a microfluidic chip 22 with the requisite micropillars 10 that have soft magnetic coatings over non-magnetic cores, can alternatively be used, and so some aspects of the present invention begin with FIG. 4B.

FIG. 4C is a view of the 4 stage series magnetic capture and release section of chip 22, during loading of MNPs into the magnetic chambers 21. The MNPs are preferably multi-layered NPs having: a superparamagnetic nano-scale core or interior layer; a passivating coating serving as a barrier against interaction with the superparamagnetic material; an electrostatically charged surface or sub-surface layer (or equivalent near field repulsive coating) and a functionalized surface adapted to selectively bond to an analyte of interest. Each of the 4 MNP loading inlets 26 is preferably loaded with MNPs having respective functionalizations to selectively bind to different analytes of interest.

The MNP fluid carrier 30 moves into the magnetic chamber 21, for example under the action of capillarity, centrifuge, or a pressure differential between the respective port and the loading inlet 26. A higher surface area of the magnetic chamber 21 as a result of the micropillars naturally improves the capillarity attraction of the MNP fluid 30, and preferably encourages a coverage of the micropillar array from edge to edge of the insert 20, which defines the boundaries of the chamber 21. Surface tension and the capillary effect may be sufficient to draw the carrier 30 over the micropillar array, for a suitable fluid, and otherwise vacuum pressure at ports of the chip may be required.

After loading, a magnetic field is applied with a permanent magnet 33 having one pole inserted within the registration slot 29, as shown in FIG. 4D. FIG. 4D shows the chip 22 with the permanent magnet 33 in place. While a permanent magnet is shown, other magnetic field generators could be used if sufficiently strong and uniform. Applicant has found that a magnetic field strength of at least 110 kA/m to produce a cloud curtain of MNPs that resists moderate transverse microfluidic flow throughput. The magnetic field has successfully been generated by a pair of permanent magnets capable of producing a uniform magnetic field across the array of MNPs. While plate magnets magnetized through thickness can provide the necessary magnetic field for most applications, equivalent distributions of magnets of different shapes with proper spatial arrangements can also be used.

FIGS. 4E,F are views of a magnetic chamber 21 of chip 22, schematically illustrating MNPs distribution before and after the magnetic field is applied. In FIG. 4E the MNP fluid 30 is distributed within the magnetic chamber 21 and the MNPs are randomly and substantially uniformly, distributed, surrounding the micropillars 10, as they would be expected to be absent a magnetic field. When the magnetic field is applied the MNPs separate from the relatively uniform distribution within the MNP fluid 30, forming a new distribution characterized in that nanoparticle cloud regions 35 are formed between the micropillars 10 (only 2 labelled) aligned with the magnetic field lines, as shown in FIG. 4F. It will be appreciated that actual MNP distributions are relatively complex, depending on distributions of various properties of the individual MNPs, and the banding of the MNPs to form these nanoparticle cloud regions 35 is provided by a strong tapering of the distribution in a direction transverse to the magnetic field lines, and depends on geometric and magnetic properties of micropillars, a field strength of the permanent magnet, and the magnetic and electrostatic properties of the MNPs. The formation of these cloud walls can be observed visually, with minor optical arrangements (e.g. at magnification of 10-500×). While the MNPs may not be controlled sufficiently to exclude all MNPs between the rows, a nanoparticle cloud region is said to form when: a MNP density at every point between two adjacent pillars of a single row (i.e. the lowest MNP density between adjacent pillars) second is at least 3× higher than the MNP density midway between two rows averaged over one second; or when a mean average density between the adjacent pillars is at least two times higher (and preferably at least one order of magnitude higher) than a mean average density midway between the rows. If there was only one row, the comparison “midway between the two rows” would be understood to be at a distance normal to the polyline equal to a mean separation of the pillars.

FIG. 4F shows a fluid flow through the magnetic chamber 21 and the fluidic streamlines crossing the nanoparticle cloud regions 35. It will be appreciated that once the MNPs form cloud regions 35, the fluid carrier 30 may be removed to the waste reservoir 24, or more preferably (to avoid contamination) out through respective detection chambers 28. A cleaning solution may optionally be injected to replace the fluid carrier 30. A prepared sample to be tested for containing an analyte of interest may be directed through the cloud regions 35 while the field is maintained. As the MNPs are sufficiently densely distributed within the clouds regions 35, a probability of interaction with the analyte, is improved over prior art MNP distributions. Furthermore, the density and favourable arrangement of the MNPs for a high capture efficiency may not require as many MNPs, as their arrangement is highly efficient.

As shown in FIG. 4G, after the sample is conducted through the cloud regions 35 (followed by any washing steps for clearing the sample that did not bind to the MNPs), the permanent magnet 33 is removed, and the distribution of MNPs relaxes to a random distribution within the magnetic chamber 21. Because the magnetic remnant induction of the micropillars 10 is low, the relaxation is fast and the MNPs have low residual magnetic bonding to the micropillars 10. Injection of a displacing fluid 36 into the loading inlets 26 (and/or evacuation of the corresponding ports of detection chambers 28) allows for the extraction of the analyte bound to MNPs for further downstream processing, which depends on the analyte and the assay performed. Optical imaging of the four detection chambers 28 is schematically illustrated in FIG. 4H. The detection methods may be optical (fluorescence, absorption, SPR, SERS), electrochemical, thermal, or colorimetric, for example.

FIG. 5A is a schematic illustration of an arrangement of micropillars 10 (of FIG. 2A) for forming a fluid dynamic array for size-selective encouragement of particles to pass between micropillars 10. This arrangement is believed to be inventive in its own right. The row of micropillars 10 is set with an angle θ_{f} with respect to a direction of flow through the chamber 21. At the same time, the flow is at an angle θₘ with respect to the magnetic field line. To the degree that the magnetic field lines are parallel to the micropillar 10 array direction, θ_{f}=θₘ, but these may diverge. The specific arrangement of spacing between the micropillars 10, angles, and micropillar shapes and diameters, collectively “select” for certain particles in the sample that are strongly encouraged to pass between the micropillars 10, where the magnetic field forces the MNPs to congregate. Size selection based on the array properties, and most particularly the row shift fraction, is generally known in the art, for example as taught by Inglis et al.²⁷ The arrangement, size and shape of the pillars in the microfluidic chamber are designed to select a critical size which determines particles that are subject to zig-zag motion (generally following stream lines), or bumped motion (systematically crosses between the micropillars at regular intervals, to deviate from the stream lines). Generally particles (or cells) larger than the critical size zig-zag and smaller particles bump.²⁶

While there are several arrangements that may make favourable use of bump arrays of magnetic coated pillars in accordance with the present invention, particular attention is drawn to arrangements of the pillars that are parallel to the stream line, or parallel to the deviated bump path that is defined by the angle. An orientation of the magnetic field substantially perpendicular to both of these will have equal probability of capturing particles above and below the critical size. An orientation of the magnetic field substantially parallel to the stream line will increase probability of binding target particles following the bump path, and an orientation of the magnetic field in the direction of the bumped path will preferentially interact with zigzagging particles, such as biological cells. As other particles size selected to paths, and the paths are not equally encouraged to pass through the MNP-dense inter-pillar spaces, particles that are not encouraged to pass do not interact with the MNPs, which can be efficient for selecting MNP interactions.

An illustrative trace 31 of a single particle as it approaches, and passes between the micropillars 10 is shown. The particle typically interacts with the array by a weaving motion as it approaches the spaces between the micropillars 10 prior to and after passing through the space between two micropillars (assuming the row shift fraction is less than ½). The weaving motion is somewhat akin to motion of a bump array of a deterministic lateral displacement array. Not only does the particle have increased probability of capture by the MNPs during the crossing, but also before and after. The typical trace 31 brings the particle much closer to stagnation points near a periphery of the micropillars than a substantially normal flow through approach. Comparatively, the zig-zagging particles which alternate between stream lines, remain preferentially directed towards the stream direction furthest from the pillars. Furthermore, during the pass through the inter-pillar space, a probability that a particle will remain within a central part of the spacing between the micropillars, where a flow is fastest and a density of the MNPs is lowest, is much greater with the substantially normal flow through approach.

It should also be noted that the trace gives a false impression that the speed of the particle is uniform. The speed of the particle is mostly determined by the flow speed of the carrier liquid, which varies according to laminar flow lines. While inertia may cause some acceleration of the particle with respect to the fluid flow during a deceleration or acceleration of the liquid nearing the pillar array, the particle will dwell near the periphery of the micropillars, and thus the time and location of the particle throughout the trace 31 is expected to offer a much higher probability of interaction, resulting in a higher capture efficiency process.

FIG. 5B schematically illustrates flow through a chamber with a staggered array of micropillars 10. Unlike the micropillars of FIG. 5A, FIG. 5B shows 4-pointed star shaped micropillar. The trace 31 is otherwise similar to FIG. 5A.

FIG. 5C schematically illustrates an arrangement of lenticular micropillars 10 (as per FIG. 2C) in four, series-connected, chambers 21, which could equally be used in the process of FIGS. 4A-H, leveraging the advantages of fluid confinement of particles, and encouraging interaction.

### Example

A magnetic capture device and apparatus, and it's fabrication has been described²⁵, the entire content of which is incorporated herein by reference, including the supplementary information material. The magnetic capture device was filled with a 500 ng/ml concentration dispersion of superparamagnetic iron-oxide core silica shell nanoparticles. For present purposes, the NPs used were equivalent to NPs available from a variety of commercial suppliers. The chamber was 30 mm×17 mm. A pair of permanent magnets was initially placed at the opposite edges of the device. The magnets were 1 cm×5 cm×10 cm at a distance of 5.8 cm from each other. They generated a substantially uniform magnetic field within the capture region. We expect the graphs showing the magnetic field along different directions (for example FIG. 2 in the supplementary information material)²⁵ to be representative of the fields produced with the present invention, except that the amplitudes are stronger with the higher powered magnets used in the present invention. The position of permanent magnets was subsequently rotated with respect to the magnetic capture device, and the configuration of the cloud region was imaged using an upright optical microscope with 20× magnification.

FIGS. 7A-E are micrograph images showing MNP distributions with varying angle and field strength of permanent magnet. As is noted in FIGS. 5C,D,E, complete cloud regions (i.e. with no visual gaps) were produced in both orientations using cylindrical micropillars in a regular lattice, with a 170 kA/m uniform magnetic field.

The obtained magnetic cloud was subsequently used for capture and release of fluorescently labeled heat-killed bacteria. Initially, a pair of permanent magnets was positioned perpendicularly to the flow within the microfluidic chamber, and the chamber was filled by flowing, at a flow rate of 25 μl/min, 50 μl volume of 500 ng/ml concentration of a dispersion of anti-listeria antibody functionalized superparamagnetic iron-oxide core silica shell nanoparticles similar to those described previously²⁵. This allowed formation of inter-pillar cloud regions throughout the microfluidic chamber. Subsequently, 1 ml volume of fluorescently labeled heat-killed Listeria monocytogenes, at a concentration of 10E4 bacteria/ml, was flowed through the microfluidic chamber at a flow rate of 100 μl/min. The flow was perpendicular to the inter-pillar regions. Following 10 minutes of the flow, the magnetic field was removed, and the captured species were released from the chamber with buffer wash for three minutes.

FIG. 8 is a panel showing 6 time-lapse fluorescence images showing (a-c) capture and (d-f) release of stained dead listeria within a generated magnetic nanoparticle cloud on the magnetic capture microfluidic device: (a) at time=0 min the nanoparticle cloud is generated within the microfluidic device, and stained dead bacteria are injected into the microfluidic chip; (b) by time t=5 min we observe a substantial increase in fluorescence intensity between Ni-coated pillars, as a result of incremental bacteria capture during sample flow; (c) by time t=10 min the fluorescence intensity strengthens demonstrating efficient bacteria capture within the nanoparticle cloud; (d) at time t=11 min, the magnetic field is removed, and captured bacteria is washed with the introduction of the wash buffer; (e) by time=12 min, the fluorescence intensity decreases as listeria is being washed away and released from the chip; (f) by time=13 min, fluorescence intensity is negligible demonstrating efficient release of listeria. Thus 2 minutes of clean buffer flow at a rate of 200 μL/min efficiently removes the MNPs and stained cells.

FIG. 9 is an image of a stained cell with the MNPs bound to its surface.

Other advantages that are inherent to the structure are obvious to one skilled in the art. The embodiments are described herein illustratively and are not meant to limit the scope of the invention as claimed. Variations of the foregoing embodiments will be evident to a person of ordinary skill and are intended by the inventor to be encompassed by the following claims.

### REFERENCES

1. H. P. Dwivedi and L.-A. Jaykus, Critical Reviews in Microbiology, 2011, 37, 40-63.
2. A. K. Bhunia, Future Microbiology, 2014, 9, 935-946.
3. B. Brehm-Stecher, C. Young, L.-A. Jaykus and M. L. Tortorello, Journal of Food Protection, 2009, 72, 1774-1789.
4. M. A. M. Gijs, F. Lacharme and U. Lehmann, Chemical Reviews, 2010, 110, 1518-1563.
5. Q. Ramadan and M. M. Gijs, Microfluidics and Nanofluidics, 2012, 13, 529-542.
6. N. Pamme and C. Wilhelm, Lab on a Chip, 2006, 6, 974-980.
7. B. Ngamsom, M. J. Lopez-Martinez, M. M. N. Esfahani, J. C. Raymond, P. Broyer, P. Patel and N. Pamme, Proc. MicroTAS 2014, San Antonio, Tex., USA, 2014, 1190-1192.
8. N. Rezlescu, V. Murariu, O. Rotariu and V. Badescu, Powder technology, 1995, 83, 259-264.
9. D. W. Inglis, R. Riehn, R. H. Austin and J. C. Sturm, Applied Physics Letters, 2004, 85, 5093-5095.
10. D. W. Inglis, R. Riehn, J. C. Sturm and R. H. Austin, Journal of Applied Physics, 2006, 99, 08K101.
11. M. Franzreb, W. Holl and C. Hoffmann, U.S. Pat. No. 6,688,473.
12. M. Takayasu, E. Maxwell and D. Kelland, Magnetics, IEEE Transactions on, 1984, 20, 1186-1188.
13. M. Bu, T. B. Christensen, K. Simstrup, A. Wolff, M. F. Hansen, Sensors and Actuators A 2008, 145-146, 430-436.
14. C. P. Gooneratne and J. Kosel, Proceedings of the Sixth International Conference on Sensing Technology (IEEE, 2012), p. 97.
15. E. Mirowski, J. Moreland, S. E. Russek and M. J. Donahue, Applied Physics Letters, 2004, 84, 1786-1788.
16. K. Smistrup, B. Kjeldsen, J. Reimers, M. Dufva, J. Petersen and M. F. Hansen, Lab on a Chip, 2005, 5, 1315-1319.
17. T. Deng, M. Prentiss and G. M. Whitesides, Applied Physics Letters, 2002, 80, 461-463.
18. X. Yu, X. Feng, J. Hu, Z.-L. Zhang and D.-W. Pang, Langmuir, 2011, 27, 5147-5156.
19. X. Yu, R. He, S. Li, B. Cai, L. Zhao, L. Liao, W. Liu, Q. Zeng, H. Wang, S.-S. Guo and X.-Z. Zhao, Small, 2013, 9, 3895-3901.
20. Y.-J. Liu, S.-S. Guo, Z.-L. Zhang, W.-H. Huang, D. Baigl, M. Xie, Y. Chen and D.-W. Pang, Electrophoresis, 2007, 28, 4713-4722.
21. S. A. Khashan and E. P. Furlani, Journal of Physics D: Applied Physics, 2013, 46, 125002.
22. J. D. Adams, U. Kim and H. T. Soh, Proceedings of the National Academy of Sciences, 2008, 105, 18165-18170.
23. S. Kim, S.-I. Han, M.-J. Park, C.-W. Jeon, Y.-D. Joo, I.-H. Choi and K.-H. Han, Analytical Chemistry, 2013, 85, 2779-2786.
24. S. Khashan, A. Alazzam and E. Furlani, Scientific Reports, 2014, 4, 5299
25. L. Malic, X. F. Zhang, D. Brassard, L. Clime, J. Daoud, C. Luebbert, V. Barrere, A. Boutin, S. Bidawid, J. Farber, N. Corneau and T. Veres, Lab on a Chip, 2015, 15, 3994-4007.
26. L. R. Huang, E. C. Cox, R. H. Austin, and J. C. Sturm, Science, 2004, 304, p. 987-990.
27. Inglis et al., Lab on Chip 2006, vol. 6, page. 655.

1. Controlling superparamagnetic nanoparticle distribution in a microfluidic chamber of a microfluidic chip, where:
at least one row of at least 3 magnetically coated pillars are provided in a wall of the chamber, the pillars having a minimum separation with neighbors of 0.2-500 μm, an aspect ratio greater than 2:1, and a mean diameter of 1-1000 μm, where a polyline connects centres of the pillars; and
a fluid is contained in the chamber surrounding the pillars, the fluid suspending superparamagnetic nanoparticles (NPs) that are self-repellant to reduce agglomeration; by:
applying a magnetic field to the chamber using magnets that are outside of the microfluidic chip, the magnetic field having a local field line that is at least 75% aligned with each segment of the polyline, wherein the NPs, pillars, and thickness of the magnetic coating of the pillars, are selected so that the NPs are substantially distributed between the pillars in that at least one of the following obtains:
a NP density at every point between two adjacent pillars of a single row is at least 50% higher than the NP density midway between two adjacent rows;
a NP density at every point between two adjacent pillars of a single row is at least 50% higher than the NP density a distance normal to the polyline equal to a mean separation of the pillars;
a mean NP density in inter-pillar spaces between adjacent pillars is at least 10 times higher than a mean NP density within the chamber;
a magnified view from a direction in which end faces of the pillars are in view, there are no visible gaps in the NP density between adjacent pillars of a single row, and visible gaps across at least 80% of the chamber away from the rows.

2. Controlling according to claim 1 wherein at least ⅓ of the NPs have a surface or subsurface coating for electrostatically, sterically, or chemically repelling like particles, and the NPs are surface functionalized to selectively bond to a target analyte.

3. Controlling according to claim 1 wherein the NPs are distributed substantially only between the pillars in that at least 80% of the NPs are retained within one or more strips centred on the polylines, with a strip thickness of twice a mean diameter of the pillars.

4. Controlling according to claim 1 wherein the pillars are coated with one of: a soft magnetic shell of thickness of 0.1-20 μm, composed of a nickel-based alloy; and a soft magnetic shell of thickness of 0.1-20 μm, composed of a nickel-based alloy coated with a gold passivation layer.

5. Controlling according to claim 1 further comprising flowing a sample fluid through the chamber across the NP distribution for NP analyte capture while the magnetic field is applied.

6. Controlling according to claim 5 wherein the wall includes at least 3 rows that form a two-dimensional Bravais lattice of the pillars, with one of the primitive vectors of the lattice being oriented at an angle between 1° and 15° with respect to the liquid flow through the chamber.

7. Controlling according to claim 6 wherein the magnetic field is oriented:
in a direction that minimizes an inter-pillar space between adjacent pillars of row;
in a direction of one of two primitive vectors of a two-dimensional Bravais lattice of defined by the at least one row; or
in a flow direction through the chamber, which is oriented at an angle between 1° and 15° with respect to one of two primitive vectors of a two-dimensional Bravais lattice of defined by the at least one row.

8. (canceled)

9. Controlling according to claim 8 wherein flushing is accomplished only with fluid dynamics, and without magnetic guidance, or a density or spatial distribution of the NPs is increased within the detection chamber by mechanical, flow, magnetic or ultrasonic filtration.

10. Controlling according to claim 5 wherein the sample fluid, after flowing through the chamber, travels through a second chamber bearing a respective wall with pillars and a fluid suspending at least one second NP distribution with NPs functionalized to selectively bond to at least one second analyte, where a single magnetic field applies fields across the chamber and the second chamber concurrently.

11. Controlling according to claim 10 wherein the chamber and second chamber are stacked horizontally on separately bonded and aligned microfluidic chips.

12. Controlling according to claim 1 where:
the pillars have a mean separation of 1-100 μm, an aspect ratio greater than 3:1, and a mean diameter of 10-300 μm;
the NPs are electrostatically charged to prevent agglomeration;
the magnetic field has a local field line that is at least 90% aligned with the segments of the polyline, and has a magnetic field strength of at least 110 kA/m across this local field line; and
during the application of the magnetic field, the NPs are distributed substantially only between the pillars in that at least 80% of the NPs are retained within one or more strips centred on the polylines, with a strip thickness of twice a mean diameter of the pillars.

13. Controlling according to claim 1 where:
the pillars have a mean separation of 20-80 μm, an aspect ratio greater than 5:1, and a mean diameter of 20-150 μm;
the NPs are electrostatically charged to prevent agglomeration;
the magnetic field has a local field line that is at least 90% aligned with the segments of the polyline, and has a magnetic field strength of at least 110 kA/m across this local field line; and
during the application, the NPs are distributed substantially only between the pillars in that at least 85% of the NPs are retained within one or more strips centred on the polylines, with a strip thickness of twice a mean diameter of the pillars.

14. Controlling according to claim 1 wherein the at least one row of at least 3 magnetically coated pillars further comprises an array having at least 2 axes, along each of which axes the pillars are arranged at least one row of at least 3 pillars with a minimum separation with neighbors of 0.2-500 μm, further comprising applying the magnetic field alternately along the axes to redistribute the NPs.

15. A microfluidic device comprising:
a microfluidic chip with at least one wall of a microfluidic chamber, the wall supporting at least one row of at least 3 micropillars, where the micropillars of the row:
are arrayed to form a polyline;
have mean diameters of 1-1000 μm;
have mean separations of 0.2-500 μm;
have aspect ratios greater than 2:1; and
are composed of a low susceptibility material coated with a soft magnetic material;
a generator adapted to apply a magnetic field of at least 110 kAmp/m across the at least one row; and
a support comprising a holder for the microfluidic chip in at least one prescribed position and orientation, and a registration feature for registering the generator in a position in which a field line of the magnetic field is at least 75% aligned with the polyline.

16. A microfluidic device according to claim 15 further comprising a sample introduction chamber, an analyte detection chamber, and a sample flush reservoir, the sample introduction chamber coupled to an ingress of the microfluidic chamber by an inlet channel, the microfluidic chamber coupled to the reservoir by an outlet channel, and the microfluidic chamber coupled to the detection chamber by a NP channel.

17. A microfluidic device according to claim 15 wherein each of the at least one wall of a microfluidic chamber, is provided as an insert into an opening within a patterned microfluidic chip.

18. A microfluidic device according to claim 15 wherein the soft magnetic coating comprises a soft magnetic shell of thickness of 0.1-20 μm, composed of a nickel-based alloy to ensure a low remanence.

19. A microfluidic device according to claim 15 wherein the microfluidic device comprises a plurality of the microfluidic chambers on one or more microfluidic chips, and the support comprises a holder for the one or more microfluidic chips in prescribed positions and orientations, and the registration feature registers the generator in a position in which one or more field lines of the magnetic field generated are at least 75% aligned with each of the respective polylines of the respective walls of the microfluidic chambers.

20. A microfluidic device according to claim 15 wherein the at least one row of at least 3 magnetically coated pillars comprises an array having at least 2 axes, along each of which axes at least one row of at least 3 pillars are arranged with a minimum separation with neighbors of 0.2-500 μm, the holder comprises a plurality of registration features for registering the generator in respective positions in which field lines of the magnetic fields are at least 75% aligned with the axes.

21. A kit comprising: the microfluidic device according to claim 15, and a fluid suspending superparamagnetic nanoparticles (NPs), the fluid being injectable into the microfluidic channel, wherein:
the NPs are self-repellant to reduce agglomeration, and
applying the magnetic field to the chamber with the magnet in registered position, with fluid in the microfluidic channel, substantially distributes the NPs between the pillars in that pillars in that at least one of the following obtains:
a NP density at every point between two adjacent pillars of a single row is at least 50% higher than the NP density midway between two adjacent rows;
a NP density at every point between two adjacent pillars of a single row is at least 50% higher than the NP density a distance normal to the polyline equal to a mean separation of the pillars;
a mean NP density in inter-pillar spaces between adjacent pillars is at least 10 times higher than a mean NP density within the chamber;
a magnified view from a direction in which end faces of the pillars are in view, there are no visible gaps in the NP density between adjacent pillars of a single row, and visible gaps across at least 80% of the chamber away from the rows.

22. A kit comprising: the microfluidic device according to claim 15, and a fluid suspending superparamagnetic nanoparticles (NPs), the fluid being injectable into the microfluidic channel, wherein the NPs:
have a surface or subsurface coating that makes at least ⅓ of the particles electrostatically or chemically repel like particles; and
are surface functionalized to selectively bond to an analyte.

23. A kit according to claim 22 wherein the microfluidic device has a plurality of microfluidic chambers, and a plurality of fluids are provided each suspending respective NP that are surface functionalized for selectively bonding to respective analytes; or the magnetic field is oriented:
in a direction that minimizes an inter-pillar space between adjacent pillars of row;
in a direction of one of two primitive vectors of a two-dimensional Bravais lattice of defined by the at least one row; or
in a flow direction through the chamber, which is oriented at an angle between 1° and 15° with respect to one of two primitive vectors of a two-dimensional Bravais lattice of defined by the at least one row.

24. (canceled)

25. A microfluidic chip insert for insertion in a microfluidic chip to form a chamber, the insert comprising at least one wall for the chamber, the wall defining at least one row of at least 3 pillars, where the pillars:
are arrayed to form a polyline;
have mean diameters (d) of 1-1000 μm;
have mean separations of 0.2-500 μm;
have aspect ratios greater than 2:1; and
comprise a soft magnetic coating; and
the polyline meets an edge of each pillar where the extent of the pillar is d or greater.

26.-27. (canceled)
